(19) United States
(12) Patent Application Publication
(10) Pub. No.: US 2007/0083171 A1
(43) Pub. Date: Apr. 12, 2007

(54) Rid-a-rash

(75) Inventor: Jon Scott Lynn, Milford, CT (US)

Correspondence Address: Jon Scott Lynn, 92 Bird Lane, Milford, CT, 06460-6815, US

(73) Assignee: Jon Scott Lynn, Milford, CT (US)

(21) Appl. No.: 11/546,094

(22) Filed: Oct. 11, 2006

Related U.S. Application Data
(60) Provisional application No. 60/726,041, filed on Oct. 12, 2005.

(51) Int. Cl. A61F 13/15

(52) U.S. Cl. 604/360

(57) ABSTRACT

This invention relates to the improvement of existing disposable diaper artwork, by providing the rash protection material already applied and covered by a removable sheet. Thus reducing the actual diapering timeframe by having the material already present. In addition to a quicker diaper timeframe, safety factors improve, due to the diapering remains hands-on. That is, the applier does not have to leave the baby briefly unattended to manipulate the rash protectant if left to be a separate device. This not only provides for a safer diaper-changing environment, but also may reduce insurance costs to both the consumer and the insurance companies.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not Applicable

### BACKGROUND OF THE INVENTION

The invention generally pertains to disposable diapers that are to be encapsulated with a rash preventative topically pre-applied and covered by a removable and disposable cover sheet. The said invention reduces rash preventative material waste and provides for a safer diaper-changing condition.

There are many related prior art references, for example, U.S. Pat. Nos. 7,060,867; 4,623,339; 2,627,858; 3,585,998 and 3,964,486 all are illustrative of such prior art.

While these units may be suitable for the particular purpose to which they address, they would not be as suitable for the purpose of the present invention, as heretofore described.

### BRIEF SUMMARY OF THE INVENTION

A principle object of the invention is to provide an improved disposable diaper with the rash preventative already applied whereby surpassing the shortcomings of the prior art devices.

Another object is to provide an improved disposable diaper with the rash preventative underneath a removable sheet in order to retain rash preventative freshness and solubility.

An additional object is to provide an improved disposable diaper that can be readily applied without removing ones hands from the baby, thereby reducing unintentional falls.

### BRIEF DESCRIPTION OF THE VIEW

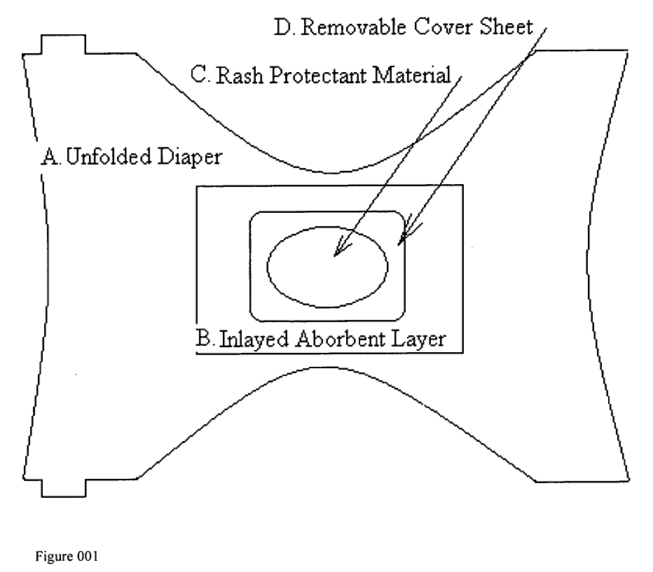

As FIG. 001 depicts, the invention is the unfolded absorbent article, for example a diaper (A), exposing the inlayed absorbent layer (B).

The rash protectant material (C) is applied on the absorbent layer (B) and is covered by the removable cover sheet (D).

### DETAILED DESCRIPTION OF THE INVENTION

As FIG. 001 depicts, the invention is the unfolded diaper (A). This diaper can be any pre-existing artwork as known to the consumer today.

The most widely used and generally accepted disposable diapers are Pampers (Proctor and Gamble) and Huggies (Kimberly Clark).

These diapers are generally designed with the inlayed absorbent layer (B). This layer is designed to absorb urine and help keep it away from the baby's skin.

The rash protectant material (C) is applied on the absorbent layer (B). The rash protectant material most widely used and generally accepted is Desitan (Pfizer) and Balmex (Johnson & Johnson).

The rash protectant material is not to be produced with any known Talc product; as such products may have been known to be produced with asbestos materials.

This rash protectant material will be covered by a removable cover sheet (D). This cover sheet will be easily and readily removable prior to completing the diapering of the baby and is disposable.

Once applied to the baby the material is to be manipulated in such a fashion to ensure coverage on the baby's bottom. Simply by patting the baby's diapered bottom will ensure such coverage and protection.

What is claimed is:

1. An improved absorbent article with which a rash deterrent solution is pre-applied.
(a) The article in claim 1 is to be primarily an infant disposable diaper.
(b) The article in claim 1 can be expanded to an adult incontinence undergarment.
(c) The article in claim 1 can also be expanded for use in the athletic undergarment industry.

2. The article in claim 1 is to be produced with a rash deterrent solution.
(a) The solution from claim 2 above is topically applied.
(b) The solution from claim 2 above is to be body facing in direction.
(c) The solution from claim 2 above is to be non-talc in composition.
(d) The solution from claim 2 is to be easily manipulated.

3. The solution from claim 2 is to be covered or encapsulated by a liner.
(a) The liner from claim 3 is to be applied so as to be easily removable.
(b) The liner from claim 3 is to be disposable.
(c) The liner from claim 3 is to be a non-permeable material.
(d) The liner from claim 3 is to prevent the rash solution from claim 2 from contacting the administrator of the absorbent article from claim 1.
